(19) United States
(12) Patent Application Publication
(10) Pub. No.: US 2012/0282343 A1
(43) Pub. Date: Nov. 8, 2012

(54) COMPOSITIONS FOR ORAL GENE THERAPY AND METHODS OF USING SAME

(75) Inventors: Kam W. Leong, Durham, NC (US); Grace Okoli, London (GB); Gonzalo Hortelano, Burlington (CA)

(73) Assignee: Johns Hopkins University, Baltimore, MD (US)

(21) Appl. No.: 13/465,410

(22) Filed: May 7, 2012

Related U.S. Application Data
(63) Continuation of application No. 10/491,544, filed on Dec. 28, 2004, now abandoned.
(60) Provisional application No. 60/326,904, filed on Oct. 3, 2001.

(51) Int. Cl. A61K 31/7088; A61P 3/00; A61K 9/14; A61K 38/02; B82Y 5/00

(52) U.S. Cl. 424/499; 514/44 R; 514/1.1; 977/773; 977/915

(57) ABSTRACT

The present invention provides nanoparticle compositions comprising a cationic biopolymer and at least one biologically active substance, pharmaceutical compositions comprising such nanoparticles and methods for the oral administration of biologically active molecules which are susceptible to degradation in the gastro-intestinal tract using nanoparticle. The present invention further provides compositions and methods for the oral administration of gene therapy.

### CROSS-REFERENCE TO RELATED APPLICATIONS

This application is a continuation of U.S. patent application Ser. No. 10/491,544, filed Dec. 28, 2004, pending, which is the U.S. national phase application pursuant to 35 U.S.C. §371 of PCT International Patent Application No. PCT/US02/31500, filed Oct. 3, 2002, which claims the benefit of U.S. provisional patent application No. 60/326,904, filed Oct. 3, 2001. The entire contents of the afore-mentioned applications are incorporated herein by reference.

### BACKGROUND OF THE INVENTION

1. Field of the Invention

The present invention generally relates to a method of delivering biologically active substances, particularly nucleic acids, into cells by oral delivery of a pharmaceutical composition comprising the biologically active substance. This invention describes a series of novel nanoparticles which comprise a cationic biopolymer and at least one biologically active substance as a delivery vehicle for oral administration of a biologically active substance which is susceptible to degradation in the gastro-intestinal tract. Preferred cationic biopolymers include chitin, chitosan and derivatives thereof. Negatively charged molecules, e.g. plasmid DNA, form complexes with these cationic biopolymers. Drugs or other biologically active substances molecules that can be delivered using these cationic lipsomes range from DNA plasmids, RNAs, peptide sequences, proteins, to small molecular weight drugs. Biodegradable nanoparticles of the invention can also be used as transport agents for genes which are orally administered to a patient.

2. Background

Effective delivery of nucleic acid to cells or tissue with high levels of expression are continued goals of gene transfer technology. As a consequence of the general inability to achieve those goals to date, however, clinical use of gene transfer methods has been limited.

Biocompatible polymeric materials have been used extensively in therapeutic percutaneous drug delivery and medical implant device applications. Sometimes, it is also desirable for such polymers to be, not only biocompatible, but also biodegradable to obviate the need for removing the polymer once its therapeutic value has been exhausted.

Conventional methods of drug delivery, such as frequent periodic dosing by percutaneous or intravenous administration, are not ideal in many cases. For example, with highly toxic drugs, frequent conventional dosing can result in high initial drug levels at the time of dosing, often at near-toxic levels, followed by low drug levels between doses that can be below the level of their therapeutic value. However, with controlled drug delivery, drug levels can be more nearly maintained at therapeutic, but non-toxic, levels by controlled release in a predictable manner over a longer term.

Oral gene delivery has many advantages when applied to replacement gene therapy. As a non-invasive procedure, repeated administration could be used to established long-term transgene expression. The term transgene refers to any gene that is delivery into a host cell using a vector delivery system.

The use of non-viral vectors in gene therapy is generally considered attractive for safety reasons and this is particularly important in Hemophilia. Up to 50% of Hemophilia patients treated prior to 1980 were infected with HIV and between 1988 and 1990 with Hepatitis, so the potential complications associated with viral gene therapy in these infected patients are a serious consideration

Long-term gene expression is the major goal for replacement gene therapy, consequently viral vectors have been a preferred delivery system in the art for use in gene therapy. Viral vectors can mediate expression over long periods of time by their stable transfection of cells but there are various safety concerns associated with viral vector. Retroviruses (RV) can integrate into the host genome and have detrimental effects on the host cell, whilst adenoviral vectors (AV) although episomal can cause aggressive immune responses that destroy cells expressing the exogenous protein and harboring the viral vector (Rosenthal, A., S. Wright, K. Quade, P. Gallimore, H. Cedar, and F. Grosveld, Increased MHC H-2K gene transcription in cultured mouse embryo cells after adenovirus infection. Nature, 1985. 315 (6020): p. 579-81). Currently, the safest and most popular viral vectors are derived from adeno-associated virus (AAV) because it is naturally replication deficient and can only replicate in the presence of an associated helper virus such as AV. Other advantages of using AAV-based vectors are; the viruses do not integrate into the host genome and has no immunogenic elements. Transduction of cells with AAV ensures stable gene expression without cytotoxic T-lymphocyte (CTL) activation but the site of injection often cause inflammation resulting in development of antibodies against the vector (Snyder, R. O., S. K. Spratt, C. Lagarde, D. Bohl, B. Kaspar, B. Sloan, L. K. Cohen, and O. Danos, Efficient and stable adeno-associated virus-mediated transduction in the skeletal muscle of adult immunocompetent mice. Hum Gene Ther, 1997. 8 (16): p. 1891-900). Other drawbacks of AAV vectors are they can only incorporate transgenes of ˜4.5 kilobases (kb) which is too small for most therapeutic gene and their regulatory regions. In addition, mass production of the rAAV has proven difficult. The most commonly used method of rAAV generation involves co-transfection of plasmids into producer cells that have already been infected with AV. So AAV purification involves the extraction of all traces of AV. Non-viral gene therapy as a safer alternative to viral vectors has been carried-out using recombinant plasmid vectors. DNA plasmid vectors have fewer safety concerns and there are no size limitations, so the genetic regulatory regions of a transgene can be included in the same construct. Plasmids can be easily manipulated for tissue-specific expression and co-expression of the transgene with desirable factors. Large-scale purification of plasmid DNA does not require helper viruses, like AAV, so it is less laborious and expensive to purify. The major disadvantages of using plasmid vectors are; transient transgene expression and low transfection efficiency. Other non-viral vector systems are naked DNA and cationic lipids. Rapid degradation of naked DNA is a problem that can be avoided by using the ‘nuclear gene gun technique’ but it is laborious and again expression is only transient. Polymers are commonly used to prolong the expression period for subcutaneously delivered or surgically inserted delivery vehicles because the recombinant vector is slowly released from the polymer. Moreover, polymers are commonly used to protect the naked DNA from in vivo degradation.

The most successful replacement gene therapy research to date has been directed towards the treatment of hemophilia B. Hemophilia B is an X-linked bleeding disorder-affecting 1 in 25,0000 individuals, it is caused by a mutation in the factor IX gene. Gene therapy for hemophilia is an attractive alternative to protein replacement therapy because continuous transgene expression would provide prophylactic protection from potentially fatal bleeds. This single gene disorder has two characteristics that deem it a good initial target for gene replacement therapy research. The first feature is most useful for viral gene delivery studies because the genes implicated in hemophilia are not regulated at the genetic level and so regulatory regions do not need to be included in the recombinant vector construct. Therefore, the overall size of the insert is much smaller than it would be if regulatory regions controlling gene expression were included. Functional activity of FIX, like all clotting factors, is governed by a series of protein interactions know as the ‘clotting cascade’, see FIG. 1. Secondly, low levels of transgene expression are adequate for therapy because in the case of hemophilia B only 1% of normal expression levels, 40-50 ng/ml of FIX in blood plasma, can be therapeutic in affected individuals. Presently replacement therapy for hemophilia entails frequent infusions of clotting factor purified from blood plasma or recombinant DNA technology techniques. With the blood purified product there is a risk of transmissible diseases such as Creutzfeld-Jakob disease and viral infections. FIX purification procedures are very expensive and as a result, most patients are treated episodically rather than prophylactically. ‘Treatment on demand’ is not an ideal strategy because there is still a risk of chronic bleeding and life threatening tissue injury. The first recombinant FIX product to be commercially available, BeneFIX®, it is manufactured using mammalian cells and in vitro transfer techniques. Problems with regulating FIX protein concentrations have been addressed by using stabilizing proteins such as human albumin but this is not an ideal method since the human albumin and remnants from the host cell line may contribute to the generation of inhibitory alloantibodies in 3% of patients.

The cost associated with FIX products for a severe hemophiliac is in excess of $100,000/year. Gene therapy for hemophilia could provide a means of prophylactic treatment by sustained replacement clotting factor expression. Dogs and mice are the two main animal models used to test the various gene therapy systems, with the aim of reproducing successful therapies in humans. Last year (1999), two research teams published gene therapy protocols for hemophilia using a hemophiliac dog model. As transgene size limitations are not a major consideration for hemophilia gene therapy these research teams used the rAAV vector to subclone FIX (1.38 kb).

Synder and coworkers (1999) showed that by delivering the gene (2×10¹²-rAAV) into the liver the site of endogenous gene expression, via the portal vein, 30-95 ng/ml of exogenous FIX expression was detected for a constant 8 month period. They showed vector dose to correlate with exogenous gene expression and functional correction. Herzog and coworkers in the same year used the intramuscular route for gene delivery injecting 6.5×10¹² viral particles per animal to achieve 40-180 ng/ml exogenous FIX levels for at least 16 months. Both groups used invasive procedures so they opted for rAAV vector delivery for stable transgene expression from a single administration.

In the past, oral gene administration has been unsuccessful, possibly because of degradation of the naked gene in the harsh conditions of the gastro-intestinal (GI) tract.

It would be desirable to provide compositions and methods which are suitable for use in oral administration of biologically active substances which are susceptible to degradation in the gastro-intestinal tract of the patient. It would be particularly desirable to provide compositions and methods of oral administration which are suitable for use in the oral delivery of genes and other DNA sequences for use in gene therapy applications.

### SUMMARY OF THE INVENTION

The present invention provides a non-invasive and safe method for long-term replacement gene therapy. This invention demonstrates that repeated gene delivery through the oral route can compensate for the transient transgene expression encountered in non-viral delivery. Long-term gene expression is the primary reason for the use of viral vectors in gene therapy, but their use may be no longer be necessary when the gene can be effectively and repeatedly administered in an oral formulation.

The present invention further provides nanoparticle compositions which comprise a cationic biopolymer and at least one biologically active substance, pharmaceutical compositions comprising same and methods of preparing and using such nanoparticle compositions to deliver biologically active substances to specified tissues or cells. In a preferred application of the present invention, nanoparticles provided by the invention are effective gene delivery agents for oral delivery of DNA to a patient being treated by gene therapy.

The present invention provides methods for oral administration of a biologically active substance which is susceptible to degradation in the gastro-intestinal tract, the method comprising the steps of:

providing an orally deliverable nanoparticle composition comprising
at least one biologically active substance susceptible to degradation in the gastro-intestinal tract; and
at least one cationic biopolymer selected from optionally substituted chitin, optionally substituted chitosan, or a derivative thereof; and

orally administering the nanoparticle composition to a patient such that at least a portion of the biologically active substance present in the nanoparticle composition is taken up by the patient without degradation in the gastro-intestinal tract.

The invention also provides methods for oral administration of a gene therapy, the method comprising the steps of:

providing an orally deliverable nanoparticle composition comprising
at least a portion of at least one gene; and
at least one cationic biopolymer selected from optionally substituted chitin, optionally substituted chitosan, or a derivative thereof; and

administering the nanoparticle composition to a patient orally such that at least a portion of gene or gene fragment present in the nanoparticle composition is delivered to a biological fluid, cell or tissue such that gene therapy occurs without degradation of the gene or gene fragment in the gastro-intestinal tract.

The invention further provides nanoparticle compositions for the oral delivery of a biologically active substance which is susceptible to degradation in the gastro-intestinal tract to a patient, the composition comprising:
at least one biologically active substance susceptible to degradation in the gastro-intestinal tract; and

at least one cationic biopolymer according to Formula II:

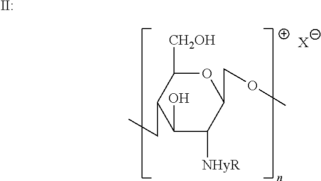

wherein

R is independently selected at each occurrence from the group consisting of hydrogen, optionally substituted alkyl, C(O)R′, steroid derivatives, and cellular recognition ligands;

R′ is independently selected at each occurrence from the group consisting of optionally substituted alkyl, steroid derivatives and cellular recognition ligands;

X is a pharmaceutically acceptable anion;

n is an integer from about 10 to about 20,000; and

y is 1 or 2.

The invention further comprises pharmaceutical compositions comprising such nanoparticles, optionally in combination with a pharmaceutically acceptable carrier.

Additional aspects of the invention are disclosed infra.

### BRIEF DESCRIPTION OF THE DRAWINGS

FIG. 1 is a schematic diagram of blood clotting cascade process;

FIG. 2 is a plot of the concentrations of hFIX in blood plasma after intravenous injection of pFIX-chitosan nanoparticles, ρFIX only and saline control. Intravenous administration of both naked DNA and nanoparticle formulations resulted in detectable hFIX plasma level;

FIG. 3 is a plot of the concentration of hFIX in blood plasma after repeated oral delivery of nanoparticles dispersed in gelatin cubes compared to intravenous injection of naked DNA (An arrow indicates each repeat administration);

FIG. 4 is a western blot using a polyclonal antibody to detect human-specific FIX expression in liver tissue taken from animals fed with pFIX nanoparticles (lane 1) and naked pFIX (lane 2);

FIG. 5a is a bar graph comparing the blood clotting time in normal mice (+/+), Factor IX knock-out mice (−/−), and mice administered with nanoparticles comprising the gene expressing Factor IX (Day 3 and Day 15); and

FIG. 5b is a plot of blood clotting times for individual mice used in the average data presented in FIG. 5a for mice administered with nanoparticles comprising the gene expressing Factor IX.

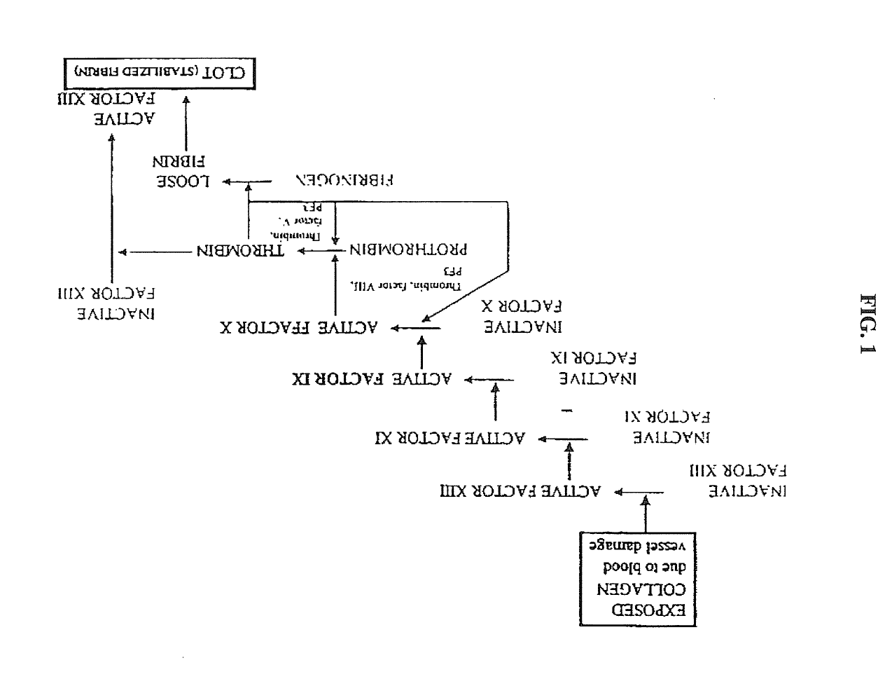

### DETAILED DESCRIPTION OF THE INVENTION

The present invention provides oral delivery methods of administering a biologically active substance which is susceptible to degradation in the gastro-intestinal tract and administration of gene therapy treatments. The present invention further provides nanoparticle compositions and pharmaceutical compositions comprising same where the nanoparticle compositions comprise a biologically active substance, including genes, which is susceptible to degradation in the gastro-intestinal tract of a patient and a cationic biopolymer.

Preferred methods of orally administering a biologically active substance which is susceptible to degradation in the gastro-intestinal tract of a patient or orally administering a gene therapy protocol include the use of nanoparticle compositions having an average particle size distribution in which the mean particle size particle size is less than a micron. More preferred methods of the invention include the use of nanoparticle compositions in which the nanoparticles have a mean particle size of between about 50 nm and about 75 nm. Preferably the minimum mean particle size of nanoparticles suitable for us in the methods of the invention is not less than about 50 nm, about 60 nm, about 70 nm, about 80 nm, about 90 nm, or about 100 nm. Preferably the maximum mean particle size of nanoparticles suitable for us in the methods of the invention is not greater than about 750 nm, about 700 nm, about 650 nm, about 600 nm, about 550 nm, 500 nm, 450 nm, 400 nm, 350 nm, 300 nm, 250 nm, or about 200 nm. Particularly preferred nanoparticle compositions suitable for use in the oral administration methods provided by the invention have a mean particle size of between about 50 nm and about 500 nm or between about 100 nm and about 250 nm.

Preferred methods of orally administering a biologically active substance which is susceptible to degradation in the gastro-intestinal tract of a patient or orally administering a gene therapy protocol include the use of nanoparticle compositions having a cationic biopolymer which has a molecular weight of between about 5 and about 2000 kDa. More preferably the molecular weight of cationic biopolymers suitable for use in the oral administration methods of the present invention are greater than about 10, about 20, about 30, about 40 or about 50 kDa and less than about 2000, about 1500, about 1250, or about 1000 kDa.

Preferred methods of orally administering a biologically active substance which is susceptible to degradation in the gastro-intestinal tract of a patient or orally administering a gene therapy are capable of delivering a therapeutically effective amount of the biologically active substance, gene or gene fragment to the patient without degradation during uptake from the gastro-intestinal tract. More preferred methods of orally administering a biologically active substance which is susceptible to degradation in the gastro-intestinal tract of a patient or orally administering a gene therapy are capable of delivering at least about 0.05%, about 0.1%, about 0.2%, about 0.3%, about 0.4%, about 0.5%, about 0.75%, about 1%, about 5%, or about 10% of the biologically active substance, gene or gene fragment to the patient without degradation during uptake from the gastro-intestinal tract. In particularly preferred methods of oral delivery, at least about 0.1%, about 0.5%, or about 1% of the biologically active substance, gene or gene fragment to the patient without degradation during uptake from the gastro-intestinal tract.

Preferred cationic biopolymers, which are suitable for use in the oral administration methods of delivering a biologically active substance or the oral administration methods of gene therapy, include those cationic biopolymers selected from cationic optionally substituted chitosan polymer which may be O- or N-substituted at some or all of the repeat units with one or more groups selected from optionally substituted alkyl, optionally substituted alkenyl, optionally substituted alkynyl, optionally substituted cycloalkyl, steroid derivatives, or cellular recognition ligands.

More preferred cationic biopolymers, which are suitable for use in the oral administration methods of delivering a biologically active substance or the oral administration methods of gene therapy, include cationic optionally substituted chitosan polymers according to Formula I

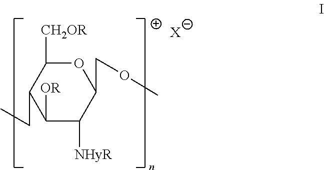

wherein

R is independently selected at each occurrence from the group consisting of hydrogen, optionally substituted alkyl, C(O)R′, steroid derivatives, and cellular recognition ligands;

R′ is independently selected at each occurrence from the group consisting of optionally substituted alkyl, steroid derivatives and cellular recognition ligands;

X is a pharmaceutically acceptable anion;

n is an integer from about 10 to about 20,000; and

y is 1 or 2.

Particularly preferred cationic biopolymers, which are suitable for use in the oral administration methods of delivering a biologically active substance or the oral administration methods of gene therapy, include cationic optionally substituted chitosan polymers according to Formula II:

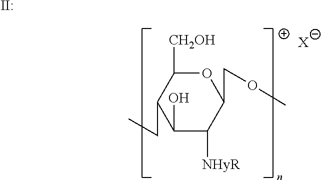

wherein

R is independently selected at each occurrence from the group consisting of hydrogen, optionally substituted alkyl, C(O)R′, steroid derivatives, and cellular recognition ligands;

R′ is independently selected at each occurrence from the group consisting of optionally substituted alkyl, steroid derivatives and cellular recognition ligands;

X is a pharmaceutically acceptable anion;

n is an integer from about 10 to about 20,000; and

y is 1 or 2.

Preferred cationic optionally substituted chitosan polymers according to Formula II include those polymers in which R is hydrogen for between about 60% and 98% of the occurrences of R in Formula II and R is C(O)R′ for between about 40% and 2% of the occurrence of R in Formula II wherein R′ is independently selected from optionally substituted lower alkyl, steroid derivatives and cellular recognition ligands.

More preferably, R is hydrogen for between about 80% and 90% of the occurrences of R in Formula II and R is C(O)R′ for between about 20% and 10% of the occurrence of R in Formula II wherein R′ is independently selected from optionally substituted lower alkyl, steroid derivatives and cellular recognition ligands.

Additional preferred cationic optionally substituted chitosan polymers according to Formula II include those polymers in which R is hydrogen for about 85% of the occurrences of R in Formula II and R is C(O)R′ for about 15% of the occurrence of R in Formula II wherein R′ is independently selected from optionally substituted lower alkyl, steroid derivatives and cellular recognition ligands

Preferred methods of orally administering a biologically active substance which is susceptible to degradation in the gastro-intestinal tract of a patient include the use of nanoparticle compositions which comprise a biologically active substance selected from the group consisting of DNA sequences, RNA sequences, peptide sequences, proteins, and small molecule therapeutics.

Preferred methods of orally administering a biologically active substance, which is susceptible to degradation in the gastro-intestinal tract of a patient or of orally administering gene therapy, include the use of nanoparticle compositions which comprise a biologically active substance, a gene or gene fragment selected from DNA sequences which express a protein in which the patient receiving treatment is deficient. Particularly preferred methods include nanoparticles comprising a biologically active substance selected from DNA sequences which encode a gene or gene fragment in which the patient receiving treatment is deficient.

Other preferred methods of orally administering a biologically active substance, which is susceptible to degradation in the gastro-intestinal tract of a patient or of orally administering gene therapy, include the use of nanoparticle compositions which are suitable for systemic delivery of the biologically active substance, gene, or gene fragment after uptake from the gastro-intestinal tract.

Yet other preferred methods of orally administering a biologically active substance, which is susceptible to degradation in the gastro-intestinal tract of a patient or of orally administering gene therapy, include the use of nanoparticle compositions which are suitable for delivery of the biologically active substance, gene, or gene fragment to a specified cell, tissue, or organ after uptake from the gastro-intestinal tract. In preferred methods of oral administration for cell, tissue, or organ specific delivery of the biologically active substance, gene, or gene therapy, at least a portion of the R groups of Formula I or II are cellular recognition ligands.

The present invention further provides methods of oral administration of gene therapy suitable for the treatment or prevention of diseases or disorders which improper expression of one or more gene sequence. Preferred methods for the oral administration of gene therapy include the use of nanoparticle compositions which comprise a gene or gene fragment which is capable of expressing a protein in which the patient receiving treatment is deficient. More preferred nanoparticle compositions suitable for use in the oral administration methods of the invention include nanoparticles which comprise a gene or gene fragment that expresses a proteing suitable for the treatment or prevention of hemophilia, metabolic disorders, hormonal disorders and the like. Particularly preferred oral administration of gene therapy methods provided by the invention are suitable for the treatment or prevention of hemophilia including hemophilia A and hemophilia B.

Suitable subjects for orally administration of gene therapy using the compositions and methods of the invention are typically mammals. Particularly preferred mammals include rodents, including mice and rats, livestock such as sheep, pig, cow and the like and primates, particularly humans, however other subjects are also contemplated as within the scope of the present invention. Further, the compositions and methods of the present invention are also suitable for in vitro gene therapy applications.

The present invention further provides nanoparticle compositions which are suitable for use in the methods of the invention for the oral delivery of a biologically active substance which is susceptible to degradation in the gastro-intestinal tract to a patient, the composition comprising:
at least one biologically active substance susceptible to degradation in the gastro-intestinal tract; and

at least one cationic biopolymer according to Formula II:

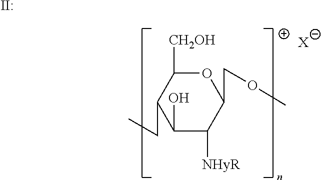

wherein

R is independently selected at each occurrence from the group consisting of hydrogen, optionally substituted alkyl, C(O)R′, steroid derivatives, and cellular recognition ligands;

R′ is independently selected at each occurrence from the group consisting of optionally substituted alkyl, steroid derivatives and cellular recognition ligands;

X is a pharmaceutically acceptable anion;

n is an integer from about 10 to about 20,000; and

y is 1 or 2.

Preferred nanoparticle compositions of the invention have an average particle size distribution in which the mean particle size particle size is less than a micron. More preferred nanoparticles have a mean particle size of between about 50 nm and about 75 nm. Preferably the minimum mean particle size of nanoparticles is not less than about 50 nm, about 60 nm, about 70 nm, about 80 nm, about 90 nm, or about 100 nm. Preferably the maximum mean particle size of nanoparticles is not greater than about 750 nm, about 700 nm, about 650 nm, about 600 nm, about 550 nm, 500 nm, 450 nm, 400 nm, 350 nm, 300 nm, 250 nm, or about 200 nm. Particularly preferred nanoparticle compositions suitable for use in the oral administration methods provided by the invention have a mean particle size of between about 50 nm and about 500 nm or between about 100 nm and about 250 nm.

Preferred nanoparticle compositions have a cationic biopolymer which has a molecular weight of between about 5 and about 2000 kDa. More preferably the molecular weight of cationic biopolymers is greater than about 10, about 20, about 30, about 40 or about 50 kDa and less than about 2000, about 1500, about 1250, or about 1000 kDa.

Preferred cationic optionally substituted chitosan polymers according to Formula II include those polymers in which R is hydrogen for between about 60% and 98% of the occurrences of R in Formula II and R is C(O)R′ for between about 40% and 2% of the occurrence of R in Formula II wherein R′ is independently selected from optionally substituted lower alkyl, steroid derivatives and cellular recognition ligands.

More preferably, R is hydrogen for between about 80% and 90% of the occurrences of R in Formula II and R is C(O)R′ for between about 20% and 10% of the occurrence of R in Formula II wherein R′ is independently selected from optionally substituted lower alkyl, steroid derivatives and cellular recognition ligands.

Additional preferred cationic optionally substituted chitosan polymers according to Formula II include those polymers in which R is hydrogen for about 85% of the occurrences of R in Formula II and R is C(O)R′ for about 15% of the occurrence of R in Formula II wherein R′ is independently selected from optionally substituted lower alkyl, steroid derivatives and cellular recognition ligands

Preferred nanoparticle compositions which comprise a biologically active substance selected from the group consisting of DNA sequences, RNA sequences, peptide sequences, proteins, and small molecule therapeutics. More preferred nanoparticle compositions comprise a biologically active substance, a gene or gene fragment selected from DNA sequences which express a protein in which the patient receiving treatment is deficient. Particularly preferred nanoparticles comprising a biologically active substance selected from DNA sequences which encode a gene or gene fragment in which the patient receiving treatment is deficient.

The present invention further provides pharmaceutical compositions comprising a nanoparticle composition of the invention and a pharmaceutically acceptable carrier.

A pharmaceutical composition of the invention also may be packaged together with instructions (i.e. written, such as a written sheet) for oral administration method disclosed herein, e.g. instruction for oral administration of a biologically active substance, gene or gene fragment which is susceptible to degradation in the gastro-intestinal tract by employing a nanoparticle composition of a cationic biopolymer and the biologically active substance, gene or gene fragment.

The present invention further provides methods of manufacturing nanoparticle compositions of the invention, the manufacturing method comprising the steps of:

providing at least one cationic biopolymer selected from optionally substituted chitin, optionally substituted chitosan, or a derivative thereof and at least one biologically active substance;

combining the cationic biopolymer and the biologically active substance in a homogeneous solution;

inducing phase separation of the homogeneous solution under conditions conducive to the formation of a nanoparticle composition comprising the cationic biopolymer and the biologically active substance.

Nucleic acid administered in accordance with the invention may be any nucleic acid (DNA or RNA) including genomic DNA, cDNA, mRNA and tRNA. These constructs may encode a gene product of interest, e.g. a therapeutic or diagnostic agent. A wide variety of known polypeptides are known that may be suitably administered to a patient in accordance with the invention.

For instance, for administration to cardiac myocytes, nucleic acids that encode vasoactive factors may be employed to treat vasoconstriction or vasospasm. Nucleic acids that encode angiogenic growth factors may be employed to promote revascularization. Suitable angiogenic growth factors include e.g. the fibroblast growth factor (FGF) family, endothelial cell growth factor (ECGF) and vascular endothelial growth factor (VEGF; see U.S. Pat. Nos. 5,332,671 and 5,219,739). See Yanagisawa-Miwa et al., Science 1992, 257:1401-1403; Pu et al., J Surg Res 1993, 54:575-83; and Takeshita et al., Circulation 1994, 90:228-234. Additional agents that may be administered to ischemic heart conditions, or other ischemic organs include e.g. nucleic acids encoding transforming growth factor α (TGF-α), transforming growth factor β (TGF-β), tumor necrosis factor α and tumor necrosis factor β. Suitable vasoactive factors that can be administered in accordance with the invention include e.g. atrial natriuretic factor, platelet-derived growth factor, endothelin and the like.

For treatment of malignancies, particularly solid tumors, nucleic acids encoding various anticancer agents can be employed, such as nucleic acids that code for diphtheria toxin, thymidinekinase, pertussis toxin, cholera toxin and the like. Nucleic acids encoding antiangiogenic agents such as matrix metalloproteases and the like also can be employed. See J. M. Ray et al. Eur Respir J 1994, 7:2062-2072.

For treatment of hemophilia including the treatment or prevention of hemophilia including treatment or prevention of hemophilia A or hemophilia B, nucleic acids including FIX genes can be employed such as Factor VII, VIII, IX and related FIX genes.

For other therapeutic applications, polypeptides transcribed by the administered nucleic acid can include growth factors or other regulatory proteins, a membrane receptor, a structural protein, an enzyme, a hormone and the like.

Also, as mentioned above, the invention provides for inhibiting expression or function of an endogenous gene of a subject. This can be accomplished by several alternative approaches. For example, antisense nucleic acid may be administered to a subject in accordance with the invention. Typically, such antisense nucleic acids will be complementary to the mRNA of the targeted endogenous gene to be suppressed, or to the nucleic acid that codes for the reverse complement of the endogenous gene. See J. H. Izant et al., Science 1985, 229:345-352; and L. J. Maher II et al., Arch Biochem Biophys 1987, 253:214-220. Antisense modulation of expression of a targeted endogenous gene can include antisense nucleic acid operably linked to gene regulatory sequences.

Alternatively, nucleic acid may be administered which antagonizes the expression of selected endogenous genes (e.g. ribozymes), or otherwise interferes with function of the endogenous gene or gene product.

The nucleic acid to be administered can be obtained by known methods, e.g. by isolating the nucleic acids from natural sources or by known synthetic methods such as the phosphate triester method. See, for example, Oligonucleotide Synthesis, IRL Press (M. J. Gait, ed. 1984). Synthetic oligonucleotides also may be prepared using commercially available automated oligonucleotide synthesizers. Also, as is known, if the nucleic acid to be administered is mRNA, it can be readily prepared from the corresponding DNA, e.g. utilizing phage RNA polymerases T3, T7 or SP6 to prepare mRNA from the DNA in the presence of ribonucleoside triphosphates. The nucleotide sequence of numerous therapeutic and diagnostic peptides including those discussed above are disclosed in the literature and computer databases (e.g., GenBank, EMBL and Swiss-Prot). Based on such information, a DNA segment may be chemically synthesized or may be obtained by other known routine procedures such as PCR.

To facilitate manipulation and handling of the nucleic acid to be administered, the nucleic acid is preferably inserted into a cassette where it is operably linked to a promoter. The promoter should be capable of driving expression in the desired cells. The selection of appropriate promoters can be readily accomplished. For some applications, a high expression promoter is preferred such as the 763-base pair cytomegalovirus (CMV) promoter. The Rous sarcoma (RSV) (Davis et al., Hum Gene Ther, 1993, 4:151) and MMT promoters also may be suitable. Additionally, certain proteins can be expressed using their native promoter. Promoters that are specific for selected cells also may be employed to limit transcription in desired cells. Other elements that can enhance expression also can be included such as an enhancer or a system that results in high expression levels such as a tat gene or a tar element. A cloning vehicle also may be designed with selective receptor binding and using the promoter to provide temporal or situational control of expression.

Typical subjects to which nucleic acid will be administered for therapeutic application include mammals, particularly primates, especially humans, and subjects for xenotransplant applications such as a primate or swine, especially pigs. For veterinary applications, a wide variety of subjects will be suitable, e.g. livestock such as cattle, sheep, goats, cows, swine and the like; poultry such as chickens, ducks, geese, turkeys and the like; and pets such as dogs and cats. For diagnostic or research applications, a wide variety of mammals will be suitable subjects including rodents (e.g. mice, rats, hamsters), rabbits, primates, and swine such as inbred pigs and the like.

An “expressible” gene is a polynucleotide with an encoding sequence, which is capable of producing the functional form of the encoded molecule in a particular cell. For a sequence encoding a polypeptide, the gene is capable of being transcribed and translated. For an anti-sense molecule, the gene is capable of producing replicate transcripts comprising anti-sense sequence. For a sequence encoding a ribozyme, the gene is capable of producing catalytic RNA.

For purposes of gene therapy, the vector will typically contain a heterologous polynucleotide of interest containing a region with a beneficial function. The polynucleotide can be directly therapeutic, but more usually will be transcribed into a therapeutic polynucleotide, such as a ribozyme or anti-sense strand, or transcribed and translated into a therapeutic polypeptide. Alternatively or in addition, the polynucleotide can provide a function that is not directly therapeutic, but which permits or facilitates another composition or agent to exert a therapeutic effect. The heterologous polynucleotide, if included, will be of sufficient length to provide the desired function or encoding sequence, and will generally be at least about 100 base pairs long, more usually at least about 200 base pairs, frequently at least about 500 base pairs, often at least about 2 kilobases, and on some occasions about 5 kilobases or more.

The effective dose of nucleic acid will be a function of the particular expressed protein, the target tissue, the subject (including species, weight, sex, general health, etc.) and the subject's clinical condition. Optimal administration rates for a given protocol of administration can be readily ascertained by those skilled in the art using conventional dosage determination tests. Additionally, frequency of administration for a given therapy can vary, particularly with the time cells containing the exogenous nucleic acid continue to produce the desired polypeptide as will be appreciated by those skilled in the art. Also, in certain therapies, it may be desirable to employ two or more different proteins to optimize therapeutic results.

The concentration of nucleic acid within a polymer nanoparticle can vary, but relatively high concentrations are preferred to provide increased efficiency of nucleic acid uptake. More specifically, preferred nanoparticles and micelles comprise a cationic biopolymer-nucleic acid complex particularly optionally substituted cationic chitosan-nucleic acid complexes and includes between about 1% to 70% by weight of the nucleic acid. More preferably, the nanoparticle comprises about 10 to about 60% nucleic acid by weight or 10%, 20%, 30%, 40%, 50% or 60% by weight of the nucleic acid.

As indicated above, various substituents of the various Formulae are “optionally substituted”, including R and R′ of Formula I and II. When substituted, those substituents may be substituted by other than hydrogen at one or more available positions, typically 1 to about 6 positions or more typically 1 to about 3 or 4 positions, by one or more suitable groups such as those disclosed herein. Suitable groups that may be present on a “substituted” R and R′ group or other substituent include e.g. halogen such as fluoro, chloro, bromo and iodo; cyano; hydroxyl; nitro; azido; alkanoyl such as a C₁₋₆ alkanoyl group such as acyl and the like; carboxamido; alkyl groups including those groups having 1 to about 12 carbon atoms, or 1, 2, 3, 4, 5, or 6 carbon atoms; alkenyl and alkynyl groups including groups having one or more unsaturated linkages and from 2 to about 12 carbon, or 2, 3, 4, 5 or 6 carbon atoms; alkoxy groups having those having one or more oxygen linkages and from 1 to about 12 carbon atoms, or 1, 2, 3, 4, 5 or 6 carbon atoms; aryloxy such as phenoxy; alkylthio groups including those moieties having one or more thioether linkages and from 1 to about 12 carbon atoms, or 1, 2, 3, 4, 5 or 6 carbon atoms; alkylsulfinyl groups including those moieties having one or more sulfinyl linkages and from 1 to about 12 carbon atoms, or 1, 2, 3, 4, 5, or 6 carbon atoms; alkylsulfonyl groups including those moieties having one or more sulfonyl linkages and from 1 to about 12 carbon atoms, or 1, 2, 3, 4, 5, or 6 carbon atoms; aminoalkyl groups such as groups having one or more N atoms and from 1 to about 12 carbon atoms, or 1, 2, 3, 4, 5 or 6 carbon atoms; carbocyclic aryl having 6 or more carbons, particularly phenyl (e.g. an Ar group being a substituted or unsubstituted biphenyl moiety); aralkyl having 1 to 3 separate or fused rings and from 6 to about 18 carbon ring atoms, with benzyl being a preferred group; aralkoxy having 1 to 3 separate or fused rings and from 6 to about 18 carbon ring atoms, with O-benzyl being a preferred group; or a heteroaromatic or heteroalicyclic group having 1 to 3 separate or fused rings with 3 to about 8 members per ring and one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl.

As used herein, the term “a positively charged or positively chargeable group” is intended to include both positively charged functional groups such as phosphonium groups, quaternary ammonium groups and other charged groups and also chargeable functional groups that can reversibly protonated to yield a positively charged group, e.g., typical chargeable groups include primary, secondary and tertiary amines, amides and other functional groups which comprise a proton acceptor and can be protonated in aqueous media at or around neutral pH.

As used herein, “alkyl” is intended to include branched, straight-chain and cyclic saturated aliphatic hydrocarbon groups including alkylene, having the specified number of carbon atoms. Examples of alkyl include, but are not limited to, methyl, ethyl, n-propyl, i-propyl, n-butyl, s-butyl, t-butyl, n-pentyl, and s-pentyl. Alkyl groups typically have 1 to about 36 carbon atoms. Typically lower alkyl groups have about 1 to about 20, 1 to about 12 or 1 to about 6 carbon atoms. Preferred lower alkyl groups are C₁-C₂₀ alkyl groups, more preferred are C₁₋₁₂-alkyl and C₁₋₆-alkyl groups. Especially preferred lower alkyl groups are methyl, ethyl, and propyl. Typically higher alkyl groups have about 4 to about 36, 8 to about 24 or 12 to about 18 carbon atoms. Preferred higher alkyl groups are C₄-C₃₆ alkyl groups, more preferred are C₈₋₂₄-alkyl and C₁₂₋₁₈-alkyl groups.

As used herein, “heteroalkyl” is intended to include branched, straight-chain and cyclic saturated aliphatic hydrocarbon groups including alkylene, having the specified number of carbon atoms and at least one heteroatom, e.g., N, O or S. Heteroalkyl groups will typically have between about 1 and about 20 carbon atoms and about 1 to about 8 heteroatoms, preferably about 1 to about 12 carbon atoms and about 1 to about 4 heteroatoms. Preferred heteroalkyl groups include the following groups. Preferred alkylthio groups include those groups having one or more thioether linkages and from 1 to about 12 carbon atoms, more preferably from 1 to about 8 carbon atoms, and still more preferably from 1 to about 6 carbon atoms. Alylthio groups having 1, 2, 3, or 4 carbon atoms are particularly preferred. Preferred alkylsulfinyl groups include those groups having one or more sulfoxide (SO) groups and from 1 to about 12 carbon atoms, more preferably from 1 to about 8 carbon atoms, and still more preferably from 1 to about 6 carbon atoms. Alkylsulfinyl groups having 1, 2, 3, or 4 carbon atoms are particularly preferred. Preferred alkylsulfonyl groups include those groups having one or more sulfonyl (SO₂) groups and from 1 to about 12 carbon atoms, more preferably from 1 to about 8 carbon atoms, and still more preferably from 1 to about 6 carbon atoms. Alylsulfonyl groups having 1, 2, 3, or 4 carbon atoms are particularly preferred. Preferred aminoalkyl groups include those groups having one or more primary, secondary and/or tertiary amine groups, and from 1 to about 12 carbon atoms, more preferably from 1 to about 8 carbon atoms, and still more preferably from 1 to about 6 carbon atoms. Aminoalkyl groups having 1, 2, 3, or 4 carbon atoms are particularly preferred.

As used herein, “heteroalkenyl” is intended to include branched, straight-chain and cyclic saturated aliphatic hydrocarbon groups including alkenylene, having the specified number of carbon atoms and at least one heteroatom, e.g., N, O or S. Heteroalkenyl groups will typically have between about 1 and about 20 carbon atoms and about 1 to about 8 heteroatoms, preferably about 1 to about 12 carbon atoms and about 1 to about 4 heteroatoms. Preferred heteroalkenyl groups include the following groups. Preferred alkylthio groups include those groups having one or more thioether linkages and from 1 to about 12 carbon atoms, more preferably from 1 to about 8 carbon atoms, and still more preferably from 1 to about 6 carbon atoms. Alkenylthio groups having 1, 2, 3, or 4 carbon atoms are particularly preferred. Preferred alkenylsulfinyl groups include those groups having one or more sulfoxide (SO) groups and from 1 to about 12 carbon atoms, more preferably from 1 to about 8 carbon atoms, and still more preferably from 1 to about 6 carbon atoms. Alkenylsulfinyl groups having 1, 2, 3, or 4 carbon atoms are particularly preferred. Preferred alkenylsulfonyl groups include those groups having one or more sulfonyl (SO₂) groups and from 1 to about 12 carbon atoms, more preferably from 1 to about 8 carbon atoms, and still more preferably from 1 to about 6 carbon atoms. Alkenylsulfonyl groups having 1, 2, 3, or 4 carbon atoms are particularly preferred. Preferred aminoalkenyl groups include those groups having one or more primary, secondary and/or tertiary amine groups, and from 1 to about 12 carbon atoms, more preferably from 1 to about 8 carbon atoms, and still more preferably from 1 to about 6 carbon atoms. Aminoalkenyl groups having 1, 2, 3, or 4 carbon atoms are particularly preferred.

As used herein, “heteroalkynyl” is intended to include branched, straight-chain and cyclic saturated aliphatic hydrocarbon groups including alkynylene, having the specified number of carbon atoms and at least one heteroatom, e.g., N, O or S. Heteroalkynyl groups will typically have between about 1 and about 20 carbon atoms and about 1 to about 8 heteroatoms, preferably about 1 to about 12 carbon atoms and about 1 to about 4 heteroatoms. Preferred heteroalkynyl groups include the following groups. Preferred alkynylthio groups include those groups having one or more thioether linkages and from 1 to about 12 carbon atoms, more preferably from 1 to about 8 carbon atoms, and still more preferably from 1 to about 6 carbon atoms. Alkynylthio groups having 1, 2, 3, or 4 carbon atoms are particularly preferred. Preferred alkynylsulfinyl groups include those groups having one or more sulfoxide (SO) groups and from 1 to about 12 carbon atoms, more preferably from 1 to about 8 carbon atoms, and still more preferably from 1 to about 6 carbon atoms. Alkynylsulfinyl groups having 1, 2, 3, or 4 carbon atoms are particularly preferred. Preferred alkynylsulfonyl groups include those groups having one or more sulfonyl (SO₂) groups and from 1 to about 12 carbon atoms, more preferably from 1 to about 8 carbon atoms, and still more preferably from 1 to about 6 carbon atoms. Alkynylsulfonyl groups having 1, 2, 3, or 4 carbon atoms are particularly preferred. Preferred aminoalkynyl groups include those groups having one or more primary, secondary and/or tertiary amine groups, and from 1 to about 12 carbon atoms, more preferably from 1 to about 8 carbon atoms, and still more preferably from 1 to about 6 carbon atoms. Aminoalkynyl groups having 1, 2, 3, or 4 carbon atoms are particularly preferred.

As used herein, “cycloalkyl” is intended to include saturated and partially unsaturated ring groups, having the specified number of carbon atoms, such as cyclopropyl, cyclobutyl, cyclopentyl, or cyclohexyl. Also included are carbocyclic ring groups with ine or more olefinic linkages between two or more ring carbon atoms such as cyclopentenyl, cyclohexenyl and the like. Cycloalkyl groups typically will have 3 to about 8 ring members.

In the term “(C₃₋₆ cycloalkyl)C₁₋₄ alkyl”, as defined above, the point of attachment is on the alkyl group. This term encompasses, but is not limited to, cyclopropylmethyl, cyclohexylmethyl, cyclohexylethyl.

As used here, “alkenyl” is intended to include hydrocarbon chains of straight, cyclic or branched configuration, including alkenylene having one or more unsaturated carbon-carbon bonds which may occur in any stable point along the chain, such as ethenyl and propenyl. Alkenyl groups typically have 1 to about 36 carbon atoms. Typically lower alkenyl groups have about 1 to about 20, 1 to about 12 or 1 to about 6 carbon atoms. Preferred lower alkenyl groups are C₁-C₂₀ alkenyl groups, more preferred are C₁₋₁₂-alkenyl and C₁₋₆-alkenyl groups. Especially preferred lower alkenyl groups are vinyl, and propenyl. Typically higher alkenyl groups have about 4 to about 36, 8 to about 24 or 12 to about 18 carbon atoms. Preferred higher alkenyl groups are C₄-C₃₆ alkenyl groups, more preferred are C₈₋₂₄-alkenyl and C₁₂₋₁₈-alkenyl groups.

As used herein, “alkynyl” is intended to include hydrocarbon chains of straight, cyclic or branched configuration, including alkynylene, and one or more triple carbon-carbon bonds which may occur in any stable point along the chain. Alkynyl groups typically have 1 to about 36 carbon atoms. Typically lower alkynyl groups have about 1 to about 20, 1 to about 12 or 1 to about 6 carbon atoms. Preferred lower alkynyl groups are C₁-C₂₀ alkynyl groups, more preferred are C₁₋₁₂-alkynyl and C₁₋₆-alkynyl groups. Especially preferred lower alkyl groups are ethynyl, and propynyl. Typically higher alkynyl groups have about 4 to about 36, 8 to about 24 or 12 to about 18 carbon atoms. Preferred higher alkynyl groups are C₄-C₃₆ alkynyl groups, more preferred are C₈₋₂₄-alkynyl and C₁₂₋₁₈-alkynyl groups.

As used herein, “haloalkyl” is intended to include both branched and straight-chain saturated aliphatic hydrocarbon groups having the specified number of carbon atoms, substituted with 1 or more halogen (for example —CᵥF_{W} where v=1 to 3 and w=1 to (2v+1). Examples of haloalkyl include, but are not limited to, trifluoromethyl, trichloromethyl, pentafluoroethyl, and pentachloroethyl. Typical haloalkyl groups will have 1 to about 16 carbon atoms, more typically 1 to about 12 or 1 to about 6 carbon atoms.

As used herein, “a steroid derivative” is defined as an optionally substituted steroid group. A steroid is defined as a group of lipids that contain a hydrogenated cyclopentanoperhydrophenanthrene ring system. Some of the substances included in this group are progesterone, adrenocortical hormones, the gonadal hormones, cardiac aglycones, bile acids, sterols (such as cholesterol), toad poisons, saponins and some of the carcinogenic hydrocarbons. Preferred steroid derivatives include the sterol family of steroids, particularly cholesterol. Particularly preferred steroid derivatives include alkylene carboxamic acid steryl esters, e.g., -alkylene-NH—CO—O-steryl.

As used herein, “alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 16 carbon atoms, more typically 1 to about 12 or 1 to about 6 carbon atoms.

Combinations of substituents and/or variables are permissible only if such combinations result in stable compounds. A stable compound or stable structure is meant to imply a compound that is sufficiently robust to survive isolation to a useful degree of purity from a reaction mixture, and formulation into an effective therapeutic agent.

As used herein, the term “aliphatic” refers to a linear, branched, cyclic alkane, alkene, or alkyne. Preferred aliphatic groups in the biodegradable amphiphilic polyphosphate of the invention are linear or branched and have from 1 to 36 carbon atoms. Preferred lower aliphatic groups have 1 to about 12 carbon atoms and preferred higher aliphatic groups have about 10 to about 24 carbon atoms.

As used herein, the term “aryl” refers to an unsaturated cyclic carbon compound with 4n+2π electrons where n is a non-negative integer, about 5-18 aromatic ring atoms and about 1 to about 3 aromatic rings.

As used herein, the terms “heterocyclic” and “heteroalicyclic” refer to a saturated or unsaturated ring compound having one or more atoms other than carbon in the ring, for example, nitrogen, oxygen or sulfur. Typical heterocyclic groups include heteroaromatic and heteroalicyclic groups that have about a total of 3 to 8 ring atoms and 1 to about 3 fused or separate rings and 1 to about 3 ring heteroatoms such as N, O or S atoms. Illustrative heterocyclic groups include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Biologically active substances of the invention can vary widely with the purpose for the composition. The active substance(s) may be described as a single entity or a combination of entities. The delivery system is designed to be used with biologically active substances having high water-solubility as well as with those having low water-solubility to produce a delivery system that has controlled release rates. The term “biologically active substance” includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment. Preferred biologically active substances include negatively charged and neutral substances. Particularly preferred biologically active substances are DNA, RNA, proteins and negatively charged or neutral therapeutic small molecules.

Non-limiting examples of useful biologically active substances include the following expanded therapeutic categories: anabolic agents, antacids, anti-asthmatic agents, anti-cholesterolemic and anti-lipid agents, anti-coagulants, anti-convulsants, anti-diarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives, hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, antigenic materials, and prodrugs.

Specific examples of useful biologically active substances from the above categories include: (a) anti-neoplastics such as androgen inhibitors, antimetabolites, cytotoxic agents, immunomodulators; (b) anti-tussives such as dextromethorphan, dextromethorphan hydrobromide, noscapine, carbetapentane citrate, and chlophedianol hydrochloride; (c) antihistamines such as chlorpheniramine maleate, phenindamine tartrate, pyrilamine maleate, doxylamine succinate, and phenyltoloxamine citrate; (d) decongestants such as phenylephrine hydrochloride, phenylpropanolamine hydrochloride, pseudoephedrine hydrochloride, and ephedrine; (e) various alkaloids such as codeine phosphate, codeine sulfate and morphine; (f) mineral supplements such as potassium chloride, zinc chloride, calcium carbonates, magnesium oxide, and other alkali metal and alkaline earth metal salts; (g) ion exchange resins such as cholestryramine; (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; (k) expectorants such as guaifenesin; (l) antacids such as aluminum hydroxide and magnesium hydroxide; (m) biologicals such as peptides, polypeptides, proteins and amino acids, hormones, interferons or cytokines and other bioactive peptidic compounds, such as hGH, tPA, calcitonin, ANF, EPO and insulin; (n) anti-infective agents such as anti-fungals, anti-virals, antiseptics and antibiotics; and (o) antigenic materials, particularly those useful in vaccine applications.

Preferably, the biologically active substance is selected from the group consisting of polysaccharides, growth factors, hormones, anti-angiogenesis factors, interferons or cytokines, DNA, RNA, proteins and pro-drugs. In a particularly preferred embodiment, the biologically active substance is a therapeutic drug or pro-drug, more preferably a drug selected from the group consisting of chemotherapeutic agents and other anti-neoplastics, antibiotics, anti-virals, anti-fungals, anti-inflammatories, anticoagulants, an antigenic materials. Particularly preferred biologically active substances are DNA and RNA sequences that are suitable for gene therapy.

The biologically active substances are used in amounts that are therapeutically effective. While the effective amount of a biologically active substance will depend on the particular material being used, amounts of the biologically active substance from about 1% to about 65% have been easily incorporated into the present delivery systems while achieving controlled release. Lesser amounts may be used to achieve efficacious levels of treatment for certain biologically active substances.

In addition, the nanoparticle compositions of the invention can also comprise additional cationic biopolymers, so long as they do not interfere undesirably with the biodegradation characteristics of the composition. Mixtures of two or more optionally substituted cationic chitosan polymers according to Formulae I and/or II may offer even greater flexibility in designing the precise release profile desired for oral administration of the complexed biologically active substance, gene or gene fragment.

Pharmaceutically acceptable carriers may be prepared from a wide range of materials. Without being limited thereto, such materials include diluents, binders and adhesives, lubricants, disintegrants, colorants, bulking agents, flavorings, sweeteners and miscellaneous materials such as buffers and adsorbents in order to prepare a particular medicated composition.

In a non-limiting illustrative embodiment, the present invention provides a non-viral transgene delivery system developed for the long-term treatment of genetic disease. Hemophilia B has been identified as an indication suitable for gene therapy. The methods and nanoparticle compositions provided by the present invention were applied to the oral administration of nanoparticles comprising a recombinant cDNA of the gene implicated in hemophilia B and a cationic chitosan polymer. More particularly a complex coacervation of the recombinant construct with chitosan, a bioploymer found in the shells of crustaceans; under specific conditions led to the formation of nanoparticles.

Chitosan is a non-toxic compound used frequently in biomedical applications such as surgical gauze and biodegradable sutures. The chitosan-DNA nanoparticles were used for prolonged transgene expression and protection of the DNA during gastro-intestinal (GI) delivery. The nanoparticles were set in a gelatin matrix to facilitate uptake by ingestion and at a given period after ingested expression of the FIX transgene released from the nanoparticles was analyzed in systemic blood and liver tissue.

This invention involves protection of the naked plasmid DNA from conditions of the GI tract as demonstrated in oral DNA vaccination applications, where the plasmid was encapsulated in a biopolymer (7. Roy, K., H. Q. Mao, S. K. Huang, and K. W. Leong, Oral gene delivery with chitosan—DNA nanoparticles generates immunologic protection in a murine model of peanut allergy [see comments]. Nat Med, 1999. 5 (4): p. 387-91; Rathmell, J. C., M. P. Cooke, W. Y. Ho, J. Grein, S. E. Townsend, M. M. Davis, and C. C. Goodnow, CD95 (Fas)-dependent elimination of self-reactive B cells upon interaction with CD4+ T. Nature, 1995.376 (6536): p. 181-4; and Dhein, J., H. Walczak, C. Baumler, K. M. Debatin, and P. H. Krammer, Autocrine T-cell suicide mediated by APO-1/(Fas/CD95) [see comments]. Nature, 1995.373 (6513): p. 438-41). Natural polymers such as chitin and gelatin have been reacted with DNA to form protective nanoparticles (Leong, K. W., H. Q. Mαo; V. L. Truong-Le, K. Roy, S. M. Walsh, and J. T. August, DNA polycation nanospheres as non-viral gene delivery vehicles. J Controlled Release, 1998. 53 (1-3): p. 183-93). The cationic properties of these biopolymers enable ionic interactions-with oppositely charged, anionic, DNA molecules in aqueous solution, a process known complex coacervation. Each polymer has its own unique characteristics based on their chemical composition and structure. Specific polymer characteristics are conveyed to the DNA-polymer nanoparticle, thereby influencing the efficiency of transfection and the rate of DNA release in vivo. Thus, the polymer used for nanoparticle formation warrants careful consideration and the present invention provides means of controlling the cationic biopolymer and more particularly the substitution pattern of chitosan polymers used in the nanoparticles and methods of oral administration provided by the invention.

In a preferred embodiment, a derivative of chitin, chitosan was investigated as a cationic biopolymer for use in nanoparticles for oral administration of gene therapy to treat hemophilia.

Chitin is a natural polysaccharide that can be found on crustacean shells and it is non-toxic. The structure of chitin is similar to cellulose found in plants except the 2-hydroxy (—OH) group of cellulose is replaced with acetamide group (C—CONH_{Z}) group resulting in a β(1->4) linkage to form a 2-acetamido-2-deoxy-D-glycopyranose based polymer [GluNAc]. Chitin is readily degraded in vivo by lysozymes, but the rate of degradation is sensitive to the degree of N-acetylation. Chitosan is derived from partially (40-98%) N-deacetylated chain of molecular weights ranging from 50-2,000 kDa and it is not as readily degraded in vivo. At 85% deactylation chitosan is degraded gradually in vivo, we chose so this form to create DNA nanoparticles for slow and controlled DNA release for prolonged transgene expression (molecular weight—39,000 kDa).

A particularly preferred cationic chitosan polymer of Formula II in which R is a mixture of H and C(O)CH₃ are prepared according to the general synthetic procedure set forth in Scheme 1.

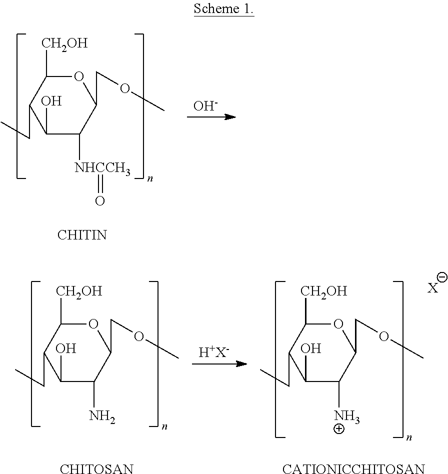

Preferred pharmaceutical compositions of the present invention have nanoparticles dispersed in a biocompatible matrix which is suitable for oral delivery of the pharmaceutical composition. Particularly preferred biocompatible matrix are composed of a non-toxic biopolymer which is subject to solvation or degradation in the gastro-intestinal tract such as starches and gelatins. A preferred non-toxic biopolymer is gelatin, which has variable physical and chemical properties depending upon the amino acids present in the gelatin sequence. Preferred gelatins for use in the pharmaceutical compositions of the present invention have as major amino acid components glycine (about 27%) and hydroxyproline (about 25%).

Collagen is the major structural protein found in animals, its denaturation by partial hydrolysis forms gelatin. Like chitosan, gelatin is non-toxic and has many uses based on its chemical and physical characteristics. Its major amino acid components are glycine (27%) and hydroxyproline (25%). The food industry uses gelatin as a gelling, stabilizer and adhesive agent. Gelatin acts as a gelling agent for the nanoparticles, facilitates easy ingestion of the pharmaceutical composition.

Repeated oral administration used in this invention a practical non-invasive method of repeated delivery to replenish transgene expression, in theory, can be performed over an indefinite period. In this invention we successfully established over 1% of basal FIX expression levels from oral delivery of the transgene expression every 4 days for an accumulative 39 days.

We demonstrated by comparing the expression kinetics of a transgene in naked DNA and DNA-nanoparticles. DNA expression was detected for longer time periods using chitosan-DNA nanoparticles. Transgene expression was detected for 21 days, using DNA nanoparticles, rather than 4 days, using naked DNA. As another alternative, cationic lipids are safe in low doses but when they form complexes with DNA, the loading levels can be low because the DNA is not efficiently condensed. Therefore, to achieve good levels of transfection high levels lipid/DNA doses are administered (Urtti, A., J. Polansky, G. M. Lui, ánd F. C. Szoka, Gene delivery and expression in human retinal pigment epithelial cells: effects of synthetic carriers, serum, extracellular matrix and viral promoters J Drug Target, 2000. 7 (6): p. 413-21). Cationic biopolymers, such as chitosan, are more effective in DNA condensation so transfection can be achieved with moderate dosage (Leong, K. W., H. Q. Mαo; V. L. Truong-Le, K. Roy, S. M. Walsh, and J. T. August, DNA polycation nanospheres as non-viral gene delivery vehicles. J Controlled Release, 1998. 53 (1-3): p. 183-93).

For successful hemophilia gene therapy the transfected tissue must be proficient in FIX synthesis and modification prior to its secretion. Liver hepatocytes are responsible for endogenous FIX synthesis and secretion, so naturally liver-specific transgene delivery would be ideal for FIX gene replacement. Other cell types capable of FIX synthesis include fibroblast, muscle and endothelial cells (Palmer, T. D., A. R. Thompson, and A. D. Miller, Production of human factor L in mammals by genetically modified sloe fibroblasts: potential therapy for hemophilia B. Blood, 1989. 73 (2): p. 438-45; Yao, S. N., J. M. Wilson, E. G. Nabei, S. Karachi, H. L. Hachiya, and K. Karachi, Express˜on of human factor IX in rat capillary endothelial cells: toward somatic gene therapy for hemophilia B. Proc Natl Acad Sci USA, 1991. 88 (18): p. 8101-5; and Yao, S. N. and K. Karachi, Express˜on of human factor IX in mice after injection of genetically modifτed myoblasts. Proc Natl Acad Sci USA, 1992. 89 (8): p. 3357-61). Intramuscular delivery of FIX has been shown to correct the functional deficiency in whole blood clotting time (WBCT) from >60 min in hemophiliac dogs to 12-20 min using 6.5×10¹² particles of AAV-FIX per dog (Snyder, R. O., C. Miao, ^{Y}L. Meuse, J. Tubb, B. A. Donahue, H. F. Lin, D. W. Stafford, S. Patel, A. R. Thompson, T. Nichols, M. S. Read, D. A. Bellinger, K. M. Brinkhous, and M. A. Kay, Correction of hemophilia B in canine and murine models using recombinant adeno-associated viral vectors. Nat Med, 1999. 5 (1): p. 64-70). Whereas liver-specific rAAV-FIX delivery of 2×10¹² particles gave a WBCT of 13-20 min, proving much more efficient than the intramuscular delivery system since it uses almost half the amount of rAAV. So, liver-specific exogenous FIX expression may generate a more functionally efficient protein than when expressed in muscle cells. Expression in the liver would also limit the potential-side effects of long-term exogenous FIX gene expression e.g. localized thrombosis. It is important to note that although our invention pertains to oral gene delivery of chitosan-DNA nanoparticles we are able to demonstrate efficient expression of the exogenous FIX protein in the liver.

During food uptake nutrients are broken-down into subunits then taken-up by either active transport or diffusion into the absorptive cells present on the mucosa of the GI tract. Once taken-up the cells nutrients undergo trans-epithelial transport into the blood stream or lymphatics. Other methods of non-specific uptake from the GI tract are paracellular transport and phagocytosis by M-cells. For liver-specific transgene expression to be detected using this invention, the chitosan-DNA nanoparticles must have been interanlized at the GI tract, probably by one or more of the described pathways. Though, the specific pathway is unknown size exclusion makes it unlikely that the nanoparticles (140 nm-200 nm) are taken-up by paracellular transport. Systemic uptake of the particle has important implications for tissue specific gene delivery and gene therapy for many genetic diseases.

Immune rejection of the exogenous protein is a major consideration for all forms of gene replacement therapy. Using this invention co-expression of the therapeutic protein with a tolerance-inducing gene, such as the Fas-ligand, in the recombinant plasmid is an option. Fas-ligand can induce apoptosis of T cells activated against the therapeutic protein as in normal T-cell development when cells recognizing self are deleted or anergized. Inhibitory antibodies detected in 3% of hemophilia B patients undergoing replacement therapy is a major consideration which may be alleviated in some patients by using a non-invasive delivery. Tissue injury caused by invasive gene delivery causes inflammation and humane response activation and such reaction can be avoid in oral delivery. Current management of inhibitory antibodies involves the daily infusion of high doses of clotting factors, resulting in 70%-90% of patients no longer producing the antibodies. Continuous expression of FIX in patients may aid in preventing inhibitory antibody, as observed in the hemophiliac dog treated with rAAV-FIX. The dog developed inhibitor antibodies that disappear without any specific treatment, process known as desensitisation.

In a non-limiting illustrative embodiment, the present invention provides a method by which long-term transgene expression can be accomplished in vivo without the need for viral vectors or invasive procedures. Using Hemophilia B as the targeted disease, exogenous FIX transgene expression was demonstrated using non-viral gene delivery in experimental mice, C57bU6 strain, by repeated oral delivery of chitosan-DNA nanoparticles containing the FIX transgene.

Encapsulation of the DNA was done to protect it from acid conditions in the stomach and enzymatic degradation in the duodenum. The vector DNA could have been of viral or non-viral origin but we chose to use a non-viral vector to avoid immune to rejection and problems associated with genome integration. The human FIX cDNA was inserted into the DNA plasmid together with two segments from FIX intron 1 to enhance expression. In theory, any type of recombinant vector could be used to form nanoparticles for oral gene delivery, for example this same invention could be used for hemophilia A gene therapy using a recombinant plasmid harboring the factor VIII gene. Properties of the plasnud must be fully considered if the system is to work efficiently: The promoter of the plasmid determines the level and mode of gene expression whether ubiquitous or tissue specific, for example if-the transgene expression is required for-liver cancer therapy expression in other tissues could be harmful and initiate unwanted function characteristics. Therefore, a liver-specific promoter should used in the recombinant construct. Also, plasmid vectors are very versatile so manipulation of the sequence to enhance gene expression (enhancer sequence inclusion) and addition of regulatory sequences is feasible because there is no size limitation, so long as the vector can be propagated for use. The plasmid vectors can be manipulated to express the exogenous gene so that tolerance of the protein by the host is achieved to enable long-term gene expression e.g. Fas-ligand co-expression with the therapeutic gene.

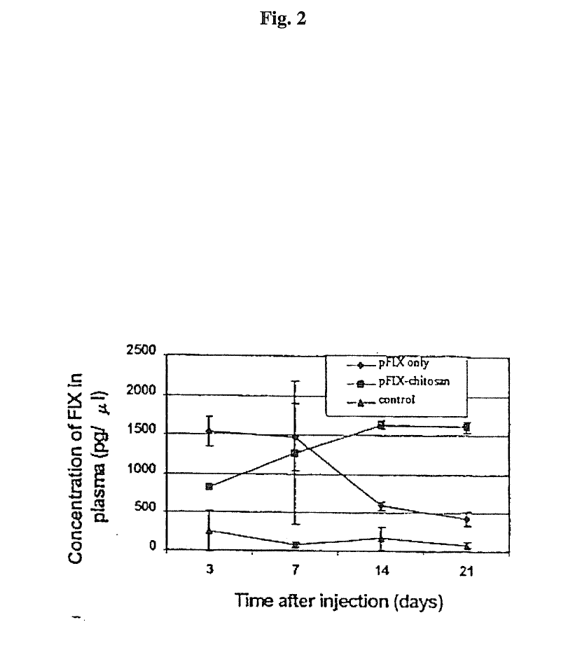

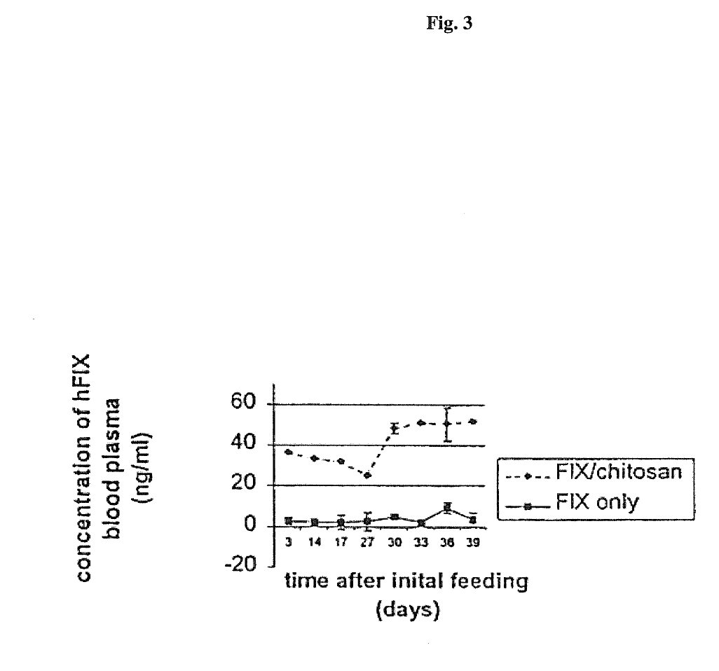

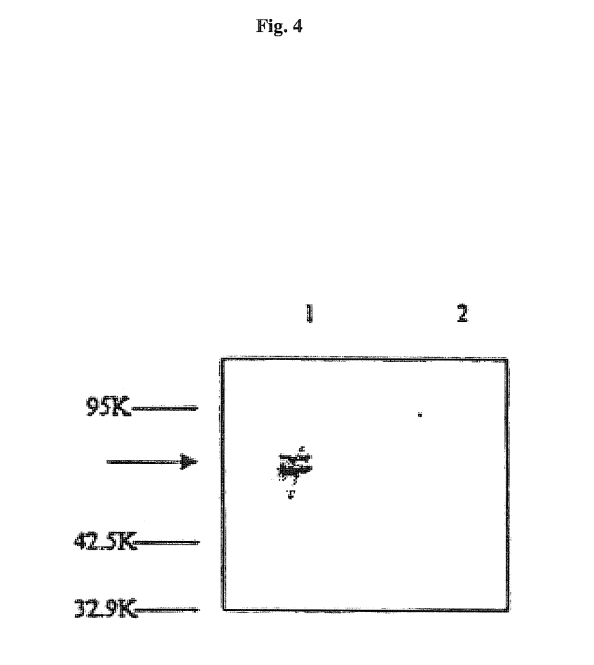

Chitosan degrades slowly in vivo and is a safe polymer to ingest. Also it has both bio-absorptive and bio-adhesive properties, making it a good cagier polymer for oral gene delivery. Any polymer with these similar characteristics could be used as the carver polymer in this invention. Controlled release of the DNA plasmid from the chitosan nanoparticles are governed by the degree of N-deactylation and the environment in which the nanoparticles are placed in vivo, in this case the GI tract. One can tailor the system so that the host ingests the nanoparticles at particular times in their feeding cycle to achieve the most effective plasmid release kinetics profile. Prolonged transgene expression was demonstrated by comparing expression kinetics of naked plasmid DNA and chitosan-DNA nanoparticles after intravenous (IV} administration in BRLB/c mice. The IV administration experiment revealed that both naked DNA and nanoparticle formulations could achieve a detectable exogenous FIX plasma level, as shown in FIG. 2. The results demonstrated a progressive increase in exogenous FIX levels over a 14 day period in chitosan-FIX-injected mice, whilst the exogenous FIX levels in mice injected with naked plasmid DNA demonstrated a gradual decline in exogenous FIX levels over the same time period. These findings would be consistent with a gradual release of the plasmid DNA from nanoparticles after entrapment in the reticulo-endothelial system, or different transfection kinetics of the nanoparticles. Either way the nanoparticles mediating prolonged periods of the transgene expression.

In FIX gene expression few proteins are able to synthesis and secrete functional FIX so liver-specific expression is preferred, inefficient expression in other cells may be harmful. We detected FIX gene expression in the liver after oral transgene delivery, indicating possible systemic transportation of the nanoparticles. Systemic nanoparticle delivery via the oral route identifies possibilities of tissue specific delivery by ligand targeting. Ligands cam be linked to chitosan nanoparticles via covalent bonding with the amine group of chitosan. A loω molecular weight ligand would be preferential used for targeted gene delivery since conjugation prior to nanoparticle formation may aid in protecting the ligand and the associated bonds from acid conditions and enzymes within the GI tract. Therefore tissue specific expression is another potential application of this invention, making it useful for all forms of gene therapy particularly gene augmentation therapy e.g. Duchenne muscular dystrophy as the defective gene is usually expressed in muscle and brain. Here a ligand conjugate such a ligand would mediated muscle specific transfection. Lack of ligand association could mediate liver delivery as demonstrated in this study.

All documents mentioned herein are incorporated herein by reference in their entirety.

The following examples are offered by way of illustration and are not intended to limit the invention in any manner.

### Example 1

### FIX Plasmid

The FIX plasmid (pFIX) construct harbored the human FIX (hFIX) cDNA sequence together with a FIX intronic sequence under the control of the beta-actin promoter and muscle creatine kinase enhancer within a Moloney murine leukemia virus backbone. By using the human FIX gene we were able to differentiate between exogenous and endogenous FIX gene expression with specific antibody based assays. In theory any vector could be used for in this invention, these days vectors with fewer viral sequences are becoming more popular for gene theory usage. Viral DNA sequences have been shown to harbor immunoreactive motifs known as CpG motifs (Krieg, A. M., Lymphocyte activation on by CpG dinucleotide motifs in prokaryotic DNA. Trends Microbiol, 1996.4 (2): p. 73-6).

### Example 2

### Generation of Plasmid-Chitosan Nanoparticles

The nanoparticles were generated by the complex coacervation of the chitosan and pFIX. Ten μg of pFIX was added to 100 μl (100 μg per ml) of 50 mM sodium sulphate and heated to 55° C. Chitosan solution, made up of 0.02% chitosan in 25 mM sodium acetate-acetic acid buffer, to solubilize the chitosan and maintain its pH during storage, was heated to 55° C. and 100 μl added to the pFIX/sodium sulphate solution while vortexed at the highest speed for 20 seconds. Sodium sulphate is used in this reaction to induce phase separation. In the acid conditions, pH 5, chitosan is highly protonated which enhances its solubility in aqueous solutions, this is necessary for the coacervation charge neutralization reaction to take place. Formation of pFIX-chitosan nanoparticles was confined using light microscopy and the particle sizes (100-200 nm) were determined by light scattering and differential interference analysis using a zetasizer (Malvern-3000). Nanoparticle size and loading levels (˜95%) are important for efficient transfect in vivo. Temperature (55° C.) and vortexing are parameters used to control the rate of coacervation and polymer size.

### Example 3

### Intravenous Delivery of Nanoparticles

Six-week old Balb/c mice were injected at the tail vein with either pFIX-chitosan (10 μg) nanoparticles, pFIX (10 μg) alone or saline (control). Four mice were IV injected in each group. The plot in FIG. 2 provides a comparison of intravenous administration of naked DNA compared to nanoparticle formulations and demonstrates a progressive increase in hFIX levels over a 14 day period in pFIX-chitosan-injected mice, whilst the hFIX levels in mice injected with naked plasmid DNA demonstrated a gradual decline in hFIX levels over the same time period. These findings would be consistent with either a gradual release of the plasmid DNA from nanoparticles after entrapment in the reticulo-endothelial system, or simply due to differences in transfection kinetics of the nanoparticles and naked DNA.

### Example 4

### Oral Delivery

Six-week old C57bU6 (Charles Rivers Breeding Labs, Wilington, Mass.) mice were fed with gelatin cubes containing 340 μl of .either pFIX-chitosan (25 μg) nanoparticles, pFIX (25 μg) solution or blank water which was added to 340 μl gelatin solution (0.083% made with water and left to set for 4 hrs at 4° C.}. Six mice were used per group.

When the mice were fed with gelatin cubes of nanoparticles comprising pFIX-chitosan (oral delivery) systemic hFIX was detectable. The levels of hFIX gradually declined over a 14-day period, in a manner similar to that observed in samples taken from mice injected with naked pFIX, see FIG. 2. Although not desiring to be bound by theory, it appears that the transfection can take place at the intestinal epithelium, as demonstrated in a previous study (Roy, K., H. Q. Mao, S. K. Huang, and K. W. Leong, Oral gene delivery with chitosan—DNA nanoparticles generates immunologic protection in a murine model of peanut allergy. Nat Med, 1999. 5 (4): p. 387-91), or the nanoparticles can be transported across the Peyer's patch and latch on to the liver or spleen. Alternatively in a Caco-2-Peyer patch model, the plasmid in the nanoparticle was observed to have-significantly degraded during the trans-epithelial transport. Notwithstanding the mode of transport of a gene or gene fragment to the tissue or organ in which expression occurs, these experiments using the C57bU6 mouse strain demonstrate the feasibility of repeated oral delivery of a gene or gene fragment using the methods and compositions of the invention. The mice were periodically fed the pFIX-chitosan nanoparticles and hFIX expression measured at 3 and 14-day intervals. The results showed the levels of hFIX were maintained above 50 ng/ml when the mice were fed at 3 day periods. With less frequent administration the systemic FIX concentration gradually declined. A gradual decline in transfection and expression efficiency was observed with subsequent administrations. This may be the result of a slight immune response, because C57bU6 mice have been shown to tolerate the human FIX better than most experimental mouse strains.

### Example 5

### Detection of Human FIX in Circulating Plasma

Human FIX was detected in blood plasma. All samples were measured in triplicate. Blood extracted from the mouse tail vein was added to 3.8% sodium citrate (9:1), to prevent blood coagulation during bleeding, and microcentrifuged at 3,000 rpm for 15 minutes to remove all cellular debris. A I:10 dilution of plasma was assayed for hFIX expression by ELISA as described by Walter and coworkers, using detection antibodies that did not cross-react with mouse FIX (Walter, J., Q. You, J. N. Hagstrom, M. Sands, and K. A. High, Successful expression of human factor IX following repeat administration of adenoviral vector in mice. Proc Natl Acad Sci USA, 1996. 93 (7): p. 3056-61). Human FIX detection in blood plasma samples was performed using 96 well plates coated with anti-FIX monoclonal antibody dilution (200 ng in 100 μl 0.1M sodium carbonate at pH9.6} and incubated at 37° C. for 2 hours. The coated plates were then blocked with ˜400 μl of blocking solution (5% slimmed milk in PBS-T, 0.04% Tween 20 in PBS) for 18 hours at 4° C. Plasma samples were diluted 1:10 in blocking solution and incubated for 1 hour at 37° C. Human FIX bound to the wells was detected by incubating the each wells for 1 hour at 37° C. using 100 μl of polyclonal human-FIX-specific primary antibody diluted in blocking solution at 1:1000. Followed by a for 1 hour at 37° C. incubation with 100 μl per well of anti-rabbit antibody conjugated with horse-radish peroxidase (HRP) diluted 1:2,500 in blocking solution. A 15 minute incubation with 100 μl of colormetric substrate, Turbo (Biorad), was stopped with 100 μl of 0.5M sulphuric acid and the absorbance measured at 450 nm. A standard reference curve was formed using human plasma with dilutions of between 0-200 ng/ml in blocking solution.

Western blots were generated from liver tissue samples taken from PBS perfused mice to determine liver-specific expression. Human FIX was specifically detected using chemilluminesense (ECL) reagent (Amersham, IL}.

Protein lysate was extracted from the liver and suspended in 5 volumes of 1% (w/v) SDS buffer were added and the tissue homogenized. The lysate suspension was sonicated twice for 30 seconds to disrupt the high molecular weight DNA and make the solution less viscous. The sample was centrifuged at 10,000 g at room temperature for 10 minutes so that the supernatant could be removed and heated at 100° C. for 10 minutes, 5 μl was used for quantification and the rest was mixed with 10× loading buffer to give a final concentration of 1×. The samples were either used immediately or stored at −80° C. The protein concentration was measured using the DC protein assay (Biorad). The reading from the standards was-taken and used to plot a graph of protein concentration against optical density, then using this graph the protein concentrations for each sample was determined from its OD at 650 nm.

100 μg of each sample was were electropharesed using a discontinuous SDS-polyacrylamide gel electrophoresis (SDS-PAGE) system and Biorad Mini Protean II slab gel apparatus. The samples were mixed with 10× loading buffer to give a concentration of 1×, heated at 100° C. for 10 minutes, placed on ice and loaded onto the discontinuous gel consisting of the upper stacking gel and lower resolving gel. Electrophoresis was performed at 100V for 60 minutes in running buffer. Samples were transferred onto a PVDF membrane using a wet transfer cell in transfer buffer at 150 mA for 1 hour. The membrane was washed with 0.4% (v/v) Tween-20 in PBS pH 7.4 for 30 minutes followed by an overnight incubation at 4° C. in blocking solution. The blot was incubated overnight at 4° C. in blocking solution containing a 1:1000 dilution of antibody or 0.5 μgml⁻¹ of the anti-human FIX polyclonal antibody (Sigma). The following day the blot was washed 5 times in 0.1% (v/v) Tween-20 in PBS. The blot was further incubated for 1 hour at room temperature in anti-rabbit IgG linked to horseradish peroxidase in a 1:2000 dilution of blocking solution. The blot was washed as before and incubated for 1 minute in ECL chemiluminescence reagent and exposed to Kodak X-Omat film for 5 seconds.

### Example 6

### Partial Correction of the Hemophilia Phenotype in Knock-Out Mice

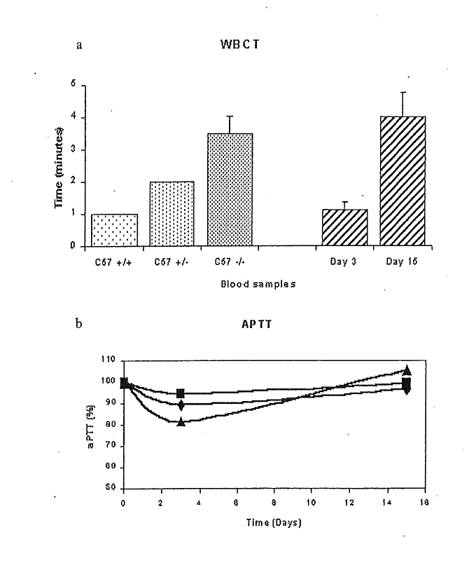

Demonstration of the bioactivity of the Factor IX transgene product was demonstrated in Factor IX knock-out mice. Shown in FIG. 6 are the blood clotting times of the control mice and mice treated with feeding of the chitosan DNA nanoparticles. The feeding protocol and DNA dose (25 μg/mouse) were the same as described in Example 4. Prior to feeding the knockout mice had a whole blood clotting time (WBCT) of 3.5 minutes compared with wild type mice which have a WBCT of 1 minute. Experimental mice fed with nanospheres displayed partial correction by a reduced clotting time of 1.3 minutes after 3 days (FIG. 5a). These mice also showed transient activated partial thromboplastin time (aPTT). The corrective phenotype was maintained for 15 days after feeding.

The mechanism of nanoparticle uptake in the GI tract could occur in two ways transfection of the intestinal epithelium, as demonstrated in a previous study (Roy, K., H. Q. Mao, S. K. Huang, and K. W. Leong, Oral gene delivery with chitosan—DNA nanoparticles generates immunologic protection in a murine model of peanut allergy. Nat Med, 1999. 5 (4): p. 387-91), or transportation across the Peyer's patch. Results from hFIX western blots show liver-specific expression in mice fed with the pFIX-chitosan nanoparticle. Each animal was perfused with PBS prior to liver extraction to ensure no systemic hFIX would be detected in the assay.

In summary, the benefits of plasmid DNA vector, in the past, has been eclipsed by transient transgene expression due to its episomal status and low tansfection efficiency when compared with viral vector delivery systems. Nanoparticles of the recombinant vector can be used to increases the expression period of the vector but not enough to mediate any long term form of therapy without repeated administration. This invention demonstrates that repeated oral administration is effective in mediating long-term transgene expression.

The invention has been described in detail with reference to preferred embodiments thereof. However, it will be appreciated that those skilled in the art, upon consideration of this disclosure, may make modifications and improvements within the spirit and scope of the invention.

1. A method for oral administration of a biologically active substance which is susceptible to degradation in the gastro-intestinal tract, the method comprising the steps of:
providing an orally deliverable nanoparticle composition comprising
at least one biologically active substance susceptible to degradation in the gastro-intestinal tract; and
at least one cationic biopolymer selected from optionally substituted chitin, optionally substituted chitosan, or a derivative thereof; and
orally administering the nanoparticle composition to a patient such that at least a portion of the biologically active substance present in the nanoparticle composition is taken up by the patient without degradation in the gastro-intestinal tract.

2. The method of claim 1, wherein the nanoparticle composition comprises a plurality of nanoparticles having an average particle size of between about 50 nm and about 500 nm.

3. (canceled)

4. The method of claim 1, wherein a therapeutically effective amount of biologically active substance present in the nanoparticle composition is taken up and/or delivered to the patient without degradation.

5-7. (canceled)

8. The method of claim 1, wherein the cationic biopolymer is a cationic optionally substituted chitosan polymer which may be O- or N-substituted at some or all of the repeat units with one or more groups selected from optionally substituted alkyl, optionally substituted alkenyl, optionally substituted alkynyl, optionally substituted cycloalkyl, steroid derivatives, or cellular recognition ligands.

9. The method of claim 1, wherein the cationic biopolymer is a cationic optionally substituted chitosan polymer according to Formula I 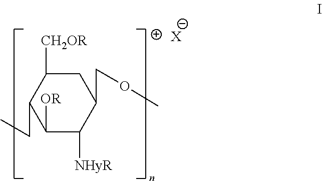
wherein
R is independently selected at each occurrence from the group consisting of hydrogen, optionally substituted alkyl, C(O)R′, steroid derivatives, and cellular recognition ligands;
R′ is independently selected at each occurrence from the group consisting of optionally substituted alkyl, steroid derivatives and cellular recognition ligands;
X is a pharmaceutically acceptable anion;
n is an integer from about 10 to about 20,000; and
y is 1 or 2.

10. The method of claim 1, wherein the cationic biopolymer is a cationic optionally substituted chitosan polymer according to Formula II: 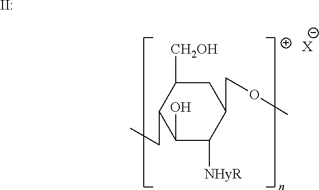
wherein
R is independently selected at each occurrence from the group consisting of hydrogen, optionally substituted alkyl, C(O)R′, steroid derivatives, and cellular recognition ligands;
R′ is independently selected at each occurrence from the group consisting of optionally substituted alkyl, steroid derivatives and cellular recognition ligands;
X is a pharmaceutically acceptable anion;
n is an integer from about 10 to about 20,000; and
y is 1 or 2.

11-13. (canceled)

14. The method of claim 1, wherein the biologically active substance is selected from the group consisting of DNA sequences, RNA sequences, peptide sequences, proteins, and small molecule therapeutics.

15-19. (canceled)

20. A method for oral administration of a gene therapy, the method comprising the steps of:
providing an orally deliverable nanoparticle composition comprising
at least a portion of at least one gene; and
at least one cationic biopolymer selected from optionally substituted chitin, optionally substituted chitosan, or a derivative thereof; and
administering the nanoparticle composition to a patient orally such that at least a portion of gene or gene fragment present in the nanoparticle composition is delivered to a biological fluid, cell or tissue such that gene therapy occurs without degradation of the gene or gene fragment in the gastro-intestinal tract.

21-26. (canceled)

27. The method of claim 20, wherein the cationic biopolymer is a cationic optionally substituted chitosan polymer which may be O- or N-substituted at some or all of the repeat units with one or more groups selected from optionally substituted alkyl, optionally substituted alkenyl, optionally substituted alkynyl, optionally substituted cycloalkyl, steroid derivatives, or cellular recognition ligands.

28. The method of claim 20, wherein the cationic biopolymer is a cationic optionally substituted chitosan polymer according to Formula II: 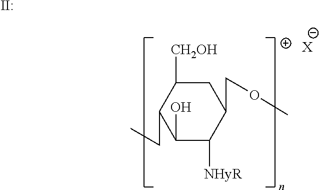
wherein
R is independently selected at each occurrence from the group consisting of hydrogen, optionally substituted alkyl, C(O)R′, steroid derivatives, and cellular recognition ligands;
R′ is independently selected at each occurrence from the group consisting of optionally substituted alkyl, steroid derivatives and cellular recognition ligands;
X is a pharmaceutically acceptable anion;
n is an integer from about 10 to about 20,000; and
y is 1 or 2.

29-31. (canceled)

32. The method of claim 20, wherein the gene or gene fragment is selected from genes or gene fragments that express a protein in which the patient receiving treatment is deficient.

33. The method of claim 20 wherein the gene or gene fragment expresses a protein suitable for the treatment of hemophilia, metabolic disorders, and hormonal disorders.

34-37. (canceled)

38. The method of claim 28, wherein at least a portion of the R groups of Formula I are cellular recognition ligands.

39-40. (canceled)

41. A nanoparticle composition for the oral delivery of a biologically active substance which is susceptible to degradation in the gastro-intestinal tract to a patient, the composition comprising:
at least one biologically active substance susceptible to degradation in the gastro-intestinal tract; and
at least one cationic biopolymer according to Formula II: 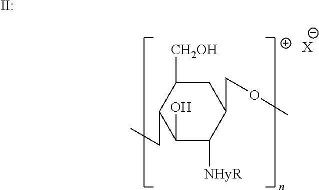
wherein
R is independently selected at each occurrence from the group consisting of hydrogen, optionally substituted alkyl, C(O)R′, steroid derivatives, and cellular recognition ligands;
R′ is independently selected at each occurrence from the group consisting of optionally substituted alkyl, steroid derivatives and cellular recognition ligands;
X is a pharmaceutically acceptable anion;
n is an integer from about 10 to about 20,000; and
y is 1 or 2.

42-49. (canceled)

50. A pharmaceutical composition comprising a nanoparticle composition of claim 41 and a pharmaceutically acceptable carrier.

51. A method of preparing a nanoparticle composition, the method comprising the steps of:
providing at least one cationic biopolymer selected from optionally substituted chitin, optionally substituted chitosan, or a derivative thereof and at least one biologically active substance;
combining the cationic biopolymer and the biologically active substance in a homogeneous solution;
inducing phase separation of the homogeneous solution under conditions conducive to the formation of a nanoparticle composition comprising the cationic biopolymer and the biologically active substance.

52-53. (canceled)
